(19) United States
(12) Patent Application Publication
(10) Pub. No.: US 2026/0340179 A1
(43) Pub. Date: Oct. 29, 2026

(54) SYSTEMS AND METHODS FOR CONSENT MANAGEMENT OF PATIENT SPECIFIC INFORMATION

(71) Applicant: Sanofi, Paris (FR)

(72) Inventors: Niranjan TUMMINKATTI, Cambridge, MA (US); Francisco Miguel ALCAÑIZ RICO, Paris (FR)

(21) Appl. No.: 19/123,082

(22) PCT Filed: Oct. 24, 2023
(86) PCT No.: PCT/IB2023/000619

§ 371 Date: Apr. 22, 2025

Related U.S. Application Data
(60) Provisional application No. 63/418,739, filed on Oct. 24, 2022.

(30) Foreign Application Priority Data
Mar. 30, 2023 (EP) 23315069.7

(51) Int. Cl. G06F 21/62; G16H 10/60; G16H 15/00

(52) U.S. Cl. CPC G06F 21/6245; G16H 10/60; G16H 15/00

(57) ABSTRACT

A non-transitory computer readable storage medium, user device and computer implemented method configured to: execute a program for managing a plurality of medical conditions, the program comprising a consent management module for managing access to patient specific information; define a plurality of user IDs and user roles within the program; assign each of the user IDs to one or more of the user roles; associate a permission level with each user role; receive a request to transmit the patient specific information to an external device, the request associated with a first user ID; and in response to receiving the request: determine the user role assigned to the first user ID; and when the permission level assigned to the user role of the first user ID permits access rights to the requested patient specific information, transmit the requested patient specific information to the external device.

### CROSS REFERENCE TO RELATED APPLICATIONS

The present application is the national stage entry of International Patent Application No. PCT/IB2023/000619, filed on Oct. 24, 2023, and claims priority to Application No. EP 23315069.7, filed on Mar. 30, 2023, and U.S. Application No. 63/418,739, filed on Oct. 24, 2022, the disclosures of which are incorporated herein by reference.

### TECHNICAL FIELD

The present specification relates to systems and methods for consent management of patient specific information, and in particular health related user data, using a plurality of roles assignable to the users of the system.

### BACKGROUND

In the field of data administration, there are many issues around data privacy and consent management to be considered. This is particularly true when the data is of a sensitive nature, such as personal health information of a patient. When building a program for assisting patients with managing their health conditions and communicating information to their healthcare professionals, the issue of consent management is an important one.

Current systems do not provide an effective means for obtaining and managing a patient's consent for their health-related data to be used in different ways. Current systems also do not provide the level of configurability that is necessary for managing consents to sensitive data.

This can negatively impact the range of functionality that the system can provide and also negatively affect patient engagement and adherence with a prescribed medication regimen as well as patient's understanding of the factors affecting their disease progression and overall health.

There is therefore a need for systems and methods with provide effective, configurable and user-friendly systems for obtaining and managing the user's consent.

### SUMMARY

A first aspect of this specification provides a computer implemented method, the method comprising:
executing a program for managing a plurality of medical conditions, the program comprising a consent module for managing access to patient specific information;
defining a plurality of user IDs within the program;
defining a plurality of user roles within the program;
assigning each of the plurality of user IDs to one or more of the plurality of user roles;
assigning a permission level to each user role, the permission level defining access rights to the patient specific information;
receiving a request to transmit the patient specific information to an external device, the request associated with a first user ID; and
in response to receiving the request:
   determining the user role assigned to the first user ID; and
   when the permission level assigned to the user role of the first user ID permits access rights to the requested patient specific information, transmitting the requested patient specific information to the external device.

The method may further comprise: generating a notification prompting the user to prepare for an upcoming appointment; and in response to receiving a user interaction with the notification, preparing a report comprising the patient specific information. The patient specific information may be health related data.

The method may further comprise revoking a permission level assigned to a first role of the plurality of user roles.

The method may further comprise: defining a time limit related to a permission level assigned to a first role of the plurality of user roles; and revoking the permission level assigned to the first role of the plurality of user roles when time limit is reached.

The method may further comprise assigning a permission level to a particular user ID of the plurality of user IDs. The method may further comprise: defining a time limit related to assigning a permission level to the particular user ID; and revoking the permission level assigned to the particular user ID when the time limit is reached.

The method may further comprise providing a consent template linking a first role of the plurality of roles with a predefined permission level.

The plurality of user roles may be selected from a list comprising at least two of: patient, physician, guardian, pharmacist and dashboard viewer. A patient may be deemed to be the sole owner of all patient specific information. The patient may be the only user that can assign consent to other users and/or roles.

The patient specific information may be categorized into different sensitivity levels.

The patient specific information may comprise a plurality of resources and the method may further comprises assigning access rights to a subset of the plurality of resources and wherein assigning a permission level may comprise granting access to the subset of the plurality of resources. A first resource of the plurality of resources may comprise information which can be used to identify the user and a second resource of the plurality of resources may comprise one or more journal entries.

Each resource may comprise a plurality of information fields. The method may further comprise granting access to a subset of the plurality of information fields within a resource. The information fields may comprise a user name, a user address, a medicament and an injection device type.

A second aspect of this specification provides a non-transitory computer readable storage medium comprising instructions that, when executed by a computer, cause the computer to:
execute a program for managing a plurality of medical conditions, the program comprising a consent management module for managing access to patient specific information;
define a plurality of user IDs within the program;
define a plurality of user roles within the program;
assign each of the plurality of user IDs to one or more of the plurality of user roles;
associate a permission level with each user role, the permission level defining access rights to the patient specific information;
receive a request to transmit the patient specific information to an external device, the request associated with a first user ID; and
in response to receiving the request:
   determine the user role assigned to the first user ID; and
   when the permission level assigned to the user role of the first user ID permits access rights to the requested patient specific information, transmitting the requested patient specific information to the external device.

The instructions may further cause the computer to: generate a notification prompting the user to prepare for an upcoming appointment; and in response to receiving a user interaction with the notification, prepare a report comprising the patient specific information.

A third aspect of this specification provides a user device comprising processing circuitry configured to:
execute a program for managing a plurality of medical conditions, the program comprising a consent management module for managing access to patient specific information;
define a plurality of user IDs within the program;
define a plurality of user roles within the program;
assign each of the plurality of user IDs to one or more of the plurality of user roles;
associate a permission level with each user role, the permission level defining access rights to the patient specific information;
receive a request to transmit the patient specific information to an external device, the request associated with a first user ID; and
in response to receiving the request:
   determine the user role assigned to the first user ID; and
   when the permission level assigned to the user role of the first user ID permits access rights to the requested patient specific information, transmit the requested patient specific information to the external device.

### BRIEF DESCRIPTION OF THE FIGURES

So that the general concepts set out in the foregoing sections can be more fully understood, embodiments thereof will be described with reference to the accompanying drawings, in which:

FIG. 1 shows an overall architecture of a system in which the computer program operates;

FIG. 2 is a high-level schematic diagram of a consent management system;

FIG. 3 is a diagram illustrating the functionality and stored information of a consent module;

FIG. 4 is a data flow diagram illustrating an application sign-up process;

FIG. 5 is a data flow diagram illustrating an “onboarding” or application customization process;

FIG. 6 is a data flow diagram illustrating steps involved in a report generation;

FIG. 7 illustrates schematically a user device for running the computer program;

FIG. 8 is a flow chart of an example method for defining and managing consent within the program for managing a plurality of medical conditions.

FIG. 9a shows a first consent screen displayed during an account creation process; and

FIG. 9b shows a privacy notice screen.

FIGS. 10a-10f show exemplary screenshots displayed by the computer program relating to facilitating user preparation for an appointment with a health care professional; and

FIGS. 11a and 11b show flow charts illustrating examples of the appointment preparation process.

### DETAILED DESCRIPTION

The present disclosure relates to an application which can be run on a user device and which provides a user consent management system for obtaining and managing the user's consent for access to their data.

The application can be used to manage a broad range of diseases which are treated by a range of different medicaments. Although some of the specific embodiments below are described in relation to the treatment of atopic dermatitis and/or asthma using a single medicament, the application is not so limited. Various other immunology indications may also be managed by the application, where one or more of these immunology indications may be treated by using a single medicament approved for use in treating the one or more immunology indications. Such a single medicament can include, for example, different dosages containing the same API, different volumes or concentrations of the same API, or different formulations containing the same API. In some embodiments, a single medicament can include an anti IL-4R mAb (e.g., Dupilumab). Further, a broad range of immunological conditions may be managed by the application. The user may be prescribed one or more drugs and the application provides personalized support for users depending on their particular drug prescription(s) and health profile. For example, the application may be configured to provide support for a number of diseases caused by Type 2 inflammation. In the context of dermatology this may include Atopic Dermatitis, Prurigo Nodularis, Bullous Pemphigoid, an Urticaria (e.g., chronic spontaneous urticaria, or cold inducible urticaria), Hand and Foot Disease, or Pruritis, each of which may be treated by injections of Dupixent® or other injectable drugs, but also by some oral and topical medications. In the context of respiratory diseases, this may include Asthma, Nasal Polyps, Sinusitis (e.g., chronic sinusitis with or without nasal polyps), Allergic Bronchopulmonary Aspergillosis (ABPA), or Allergic Fungal Rhinosinusitis (AFRS) (which may be treated by injections of Dupixent® or other injectable drugs but also by some oral medications) and Chronic Obstructive Pulmonary Disease (COPD), which may be treated by injections of Dupixent® or other injectable drugs. In the context of Gastroenterology this may include Inflammatory Bowel Disease (IBD) Eosinophilic Esophagitis (EoE), and Eosinophilic Gastroenteritis (EGE), or Ulcerative Colitis, which may be treated by injections of Dupixent® or other injectable drugs but also by some oral medications.

The medicament or medicaments may be administered by injection. As used herein, the term injection or self-injection is intended to encompass intra-venous injection, intra-muscular injection, infusion, or any needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

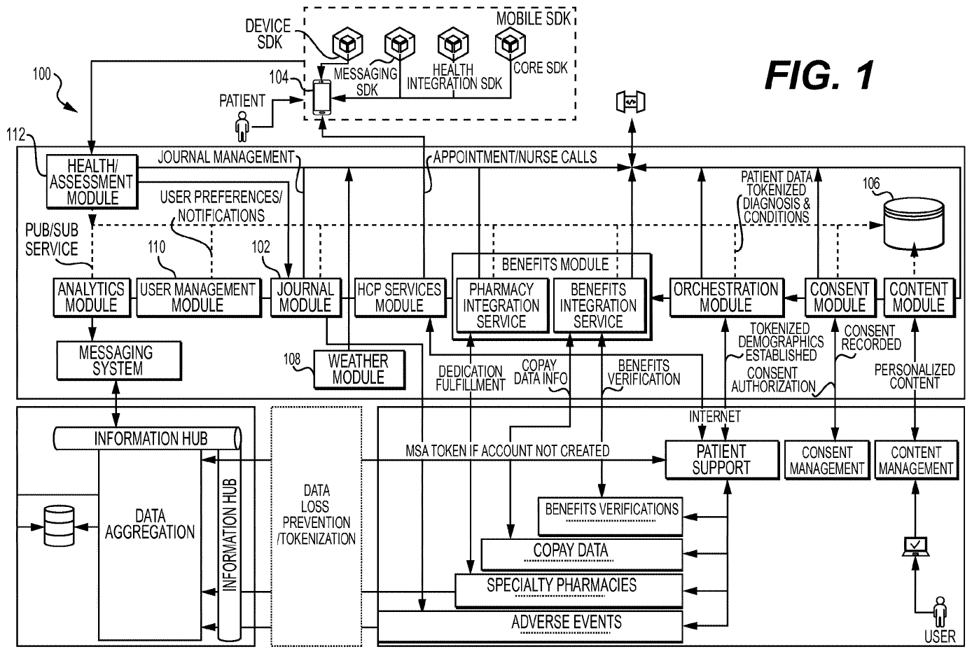

Referring to FIG. 1, an overall architecture 100 for a disease management and medication regimen management system is shown. The system architecture 100 illustrates a number of functional modules and the data links between these. The system architecture 100 comprises a user device 104 running an application with a number of functional modules, illustrated in the central box. The architecture 100 also illustrates how the application run by the user device 104 can interact with other service providers to enhance the information which can be provided to the user through the application. For instance, by communicating with insurance/benefit provider systems and pharmacies.

One of the functional modules is a journal module 102. The journal module 102 is configured to allow a user to maintain a journal relating to one or more medical conditions of the user. The journal may comprise a plurality of journal entries that detail episodes of the one or more medical conditions, such as symptoms, user specific health data, contextual information, or the like. The journal may be used to monitor the one or more medical conditions for patterns, and to identify potential triggers for episodes of the one or more medical conditions. The journal module 102 may communicate with an external service called adverse events 134. The journal module may transmit patient information associated with episodes of the one or more medical conditions to the adverse events 134 service.

The journal may further maintain a record of the adherence of the user to a treatment regimen, such as a medicament regime. For instance, the journal entry can include a record logging a dose of medicament (e.g. a scheduled dose) that has been taken by the user, such as an injection log. The record can form a dose record including a variety of information associated with the dose administered by the user. The record can be stored as part of the journal entry or independently from the journal entry.

One of these functional modules is a consent module (also referred to herein as a consent management module) which, in conjunction with other aspects of the system, is responsible for managing consents and permissions for accessing patient specific information, such as health related data for the user.

The functional modules may further comprise a memory module 106. The memory module 106 is configured to store user ID and role information as well as consent templates for use by the Consent Module. The Memory Module 106 may also store consent and permission information input by the user via the Consent Module, including any time limits set on the consents and permissions. The Memory Module 106 may, in some embodiments, be linked to the cloud, and store the information remotely.

The Consent Module may communicate with an external service called consent management service. The consent and permission information generated by the consent module may be stored or copied to the consent management service. The consent management service may also be responsible for sending the patient specific information to third parties when the associated consents indicate that this is permitted.

The Memory Module 106 may be configured to store user ID and role information as well as consent templates for use by the Consent Module 118.

Another of the functions modules is a Content Module. The content module 122 is responsible for receiving and recording personalised content and/or preferences set by the user. The content module may communicate with an external service called Content Management. The content management service responsible for providing patient specific content or settings from an external user, such as a healthcare professional. The content module may interact with the memory module 106 to store the personalised content and/or settings.

Another of the functional modules is a Benefits Module which, in conjunction with other aspects of the system, is responsible for managing patient access to health care. The Benefits Module may include a pharmacy integration service and/or a benefits integration service. The information generated by the benefits module may be responsible for sending patient specific information to third parties to indicate patient access to health care services. The benefits module may interact with external services, such as benefit provider systems and pharmacy services.

Another of the functional modules is an Analytics Module. The analytics module is responsible for analysing the patient information associated with the status and/or treatment of the user's medical conditions.

Another of the functional modules is a Messaging System. The Messaging System may communicate with the analytics module. For instance, if the analytics module identifies an episode or a trigger to an episode of the one or more medical conditioners of the user, the messaging system outputs a message communicating this result. The messaging system may communicate with an external service called an Information Hub. The information hub may store the data received from the messaging system. The information hub may also interact with other service providers to enhance the information which can be stored in respect of the user through the application. For instance, by communicating with the patient support program, insurance/benefit provider systems and pharmacies 130, adverse events services, and so on. In some embodiments, the functional modules may further comprise a weather module 108.

The weather module 108 is configured to determine and/or record weather conditions at the location of the user. The user location can be determined, for example, by a GPS capability of the user device. The weather module 108 may, for example, access an online weather forecasting system to obtain current weather conditions, previous weather conditions and/or predicted future weather conditions. The weather module 108 may provide weather conditions to the journal module 102 for inclusion in journal entries. As well as weather conditions such as general weather condition, temperature, humidity, air pressure and UV index, the weather module may additionally obtain air quality information such as pollen count, pollution index, NO₂ count and/or an air quality index.

Another of the functional modules is a user management module 110. The user management module 110 is responsible for obtaining user preferences and for generating notifications for output on the user device 104. Necessary data for the notification scheme may be stored on the memory module 106. The user management module 110 may also update the information held in the memory module 106 as the application is used.

The user management module 110 may define and help implement a notification scheme which determines when a notification should be generated and how it should be output on the user device 104. The notification scheme may have three main notification types; (i) a silent notification; (ii) a batched summary notification; and (iii) a push notification.

In some embodiments, the silent notification may be pushed into the application silently and viewable only within the application. The user may be presented with the silent notification within the application, for example on an application home screen. In some embodiments, the silent notifications are surfaced to the user when certain requirements are met and do not require any action on user's part. The batched summary notification may relate to several different reminders and other notifications. The user can interact with the batched summary notification to expand and show the individual notifications. The batch summary notification may indicate the number of notifications which are present for the user to review. The push notification may relate to a single notification and is pushed to the lock screen or home screen of the user device 104. In some embodiments, the push notifications are time-sensitive and actionable items that require the user's immediate attention. They are reserved for actions related to the user's treatment routine or as a follow up to actions that can impact the user's access to the treatment/drug.

Notifications can escalate, i.e. move up the notification types, depending on the user's action (or lack thereof). For example:
a. When the user is 5 days away from their next medicament dose, their next dosing reminder will show up as a silent notification in the application, for example underneath the carousel on the App homepage.
b. In the days leading up to the dosing, the application will push batch notifications to the user to either review instructional videos, book a call with a nurse, or review the instructions for self-administration.
c. On the day of the dosing and an hour prior to the scheduled time, the application will push a notification to the user to take their dose. If the user's medication is kept in the fridge, the notification will instruct the user to take their medicament out of the fridge. Interacting with this notification will trigger a warm-up timer, set to their medication dose.

Another of the functional modules is a Health/Assessment Module 112. The Health/Assessment Module 112 is responsible for onboarding the user and obtaining details regarding the user's medical conditions and medicaments being used to treat these. The Health/Assessment Module 112 may also gather information about the user's prescriptions and manage delivery of medicaments.

The architecture 100 also illustrates how the application run by the functional modules can interact with other service providers to enhance the information which can be provided to the user through the application, such as by communicating with insurance/benefit provider systems and pharmacies.

Another of the functional modules is a Health Care Professional (HCP) Services Module. The HCP Services Module is responsible for facilitating contact with a nurse, doctor or other health care professional, including scheduling reminders relating to appointments and calls, prompting the user to create reports or otherwise prepare for an upcoming appointment or call, initiating voice or video calls, providing follow-up notifications after calls or appointments and initiating a journal entry after calls or appointments. The HCP Services Module interacts with an internet-based Patient Support Program. Health care professionals may also have access to certain aspects and information held by the Patient Support Program, in order to facilitate contact with the user and monitor the user's treatment and/or disease progression.

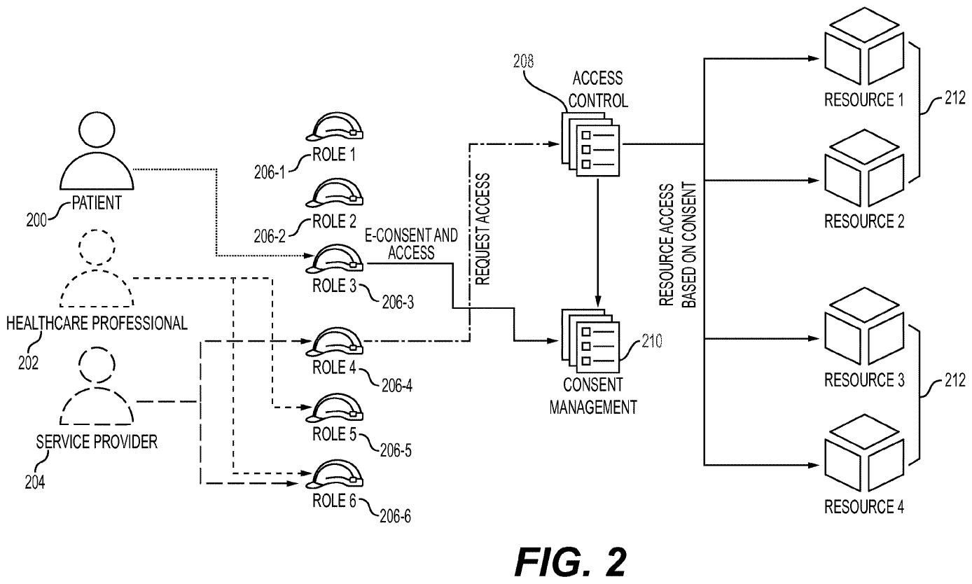

FIG. 2 is a high-level schematic diagram of a consent management system, which may be defined in and controlled by the consent module 102 and/or consent management service 108. On the left of the diagram three different types of users are shown. These are a patient 200, Healthcare Professional 202 and Service provider 204. These are given merely as examples of the types of users which may be defined in the system and does not represent an exhaustive list. The service provider 204 can include, for example, a legal manufacturer (of the application), a pharma/biotech company, a pharmacy, an insurance company, or other organization having an interest in tracking or controlling information, permissions, or other aspects of providing patient support. In some systems, the legal manufacturer of the application may also be the manufacturer of the medicament. In some other embodiments, the manufacturer of the medicament may be included as a separate user, or as a user having the same role options as the legal manufacturer. Each of the different user types is assigned a user ID, of any suitable format, identifying the user and/or user type. The user IDs are stored in the consent module 102 and/or consent management service 108. Further user types may then be added to the system by updating the information in the consent management service 108 and/or by pushing updated information to the consent module 102.

The consent management system also defines a number of different roles 206. These are labelled in FIG. 2 as Role 1 (206-1), Role 2 (206-2), Role 3 (206-3), etc. In the depicted embodiment, the patient is assigned to Role 3 (206-3). Role 3 may be one of several roles which is authorized to give consent for data to be stored or shared and to set permission levels for other roles. In some other embodiments a “patient” role is only assignable to the registered user of the application. The patient role can give consent for data to be stored or shared and set permission levels for other roles. Roles 1 and 2 may be reserved for example for user IDs assigned to “parent” or “legal guardian”. These roles may also be able to give consent for data to be stored or shared and set permission levels for other roles.

Both the Healthcare Professional 202 and Service provider 204 are assigned to two different roles each. The Healthcare Professional 202 is assigned to Role 5 (206-5) and Role 6 (206-6) while the Service provider 204 is assigned to Role 4 (206-4) and Role 6 (206-6). The user roles may for example include: patient, physician, parent, guardian, pharmacist, patient support program and dashboard viewer, among others. For example, the provider of the application may be assigned to the dashboard viewer role. The permissions associated with the dashboard viewer role may allow the provider of the application to view anonymized analytic data on the user of the application, but will not allow access to more sensitive patient information. As a further example, a service provider who provides patient support may be assigned to the patient support program role. This role may have access to more sensitive patient data which allows it to identify individual patients and access at least some of their health data. Certain types of user may not be able to be assigned to certain roles.

A pharma/biotech company, for example the manufacturer of the medicament being used by the patient, may have a number of defined entities assigned to different roles. Some entities associated with the pharma/biotech company may be assigned as a dashboard viewer only, while other entities may be assigned to a role with greater permissions, similar to that of a pharmacist.

In some embodiments, by default all roles other than the “patient” role may have minimal permissions, especially relating to sensitive patient information. More generalized information such a demographic info about a patient may be available by default to some roles. As an example, the pharmacist role may be assigned to a dispensing pharmacy. The dispensing pharmacy requires information identifying the patient, their prescription and optionally the patient's address. The pharmacist role may therefore have the appropriate permissions set by default. The patient can then assign the pharmacist role to their dispensing pharmacy using the application. The user's explicit consent may be required for such information to be sent by the application to the dispensing pharmacy and this consent may be obtained within the app during a sign-up process or when the user assigns the pharmacy role to the dispensing pharmacy.

As depicted, a user assigned to Role 4 may request access to some user specific data via an access control element 208 of the system. The access control element 208 queries the consent management element 210 (which may be the consent module 102 and/or consent management service 108) to check if Role 4 has permission to access the requested information. The requested information may be one of a number of different resources 212 and the access control element 208 controls access to these resources 212. For example, one resource may comprise information which can be used to identify the user, and may contain a number of fields such as “User name”, “User address”, “User phone number” and “User email address”.

Alternatively, the user's phone number and email address may be included in a separate resource comprising information which can be used to contact the user. Another resource may comprise health related information for the user, and may comprise a number of fields such as “Medicament”, “Injection device type”, “Dosage volume”, “Dosage concentration”, “User first health condition” and “User second health condition”. The name and address of the user's doctor may also be included in the health related information resource and/or in the contact details resource, or in a separate resource. In some other embodiments, all of the user's personal information, including information which can be used to identify them, contact details and health related information are part of a single resource.

Consents may be granted or withheld in relation to an entire resource 212, or to specific fields within a resource. Alternatively, or in addition, consent may be granted to a combination of fields selected from different resources. For example, consent may be granted to access the “User name”, “User address”, “Medicament” and “Injection device type” fields, for example to facilitate delivery of the correct medicament to the user, but consent to access the “User first health condition” and “User second health condition” may be withheld.

Further examples of resources 212 defined in the system include: written journal entries, health condition summary reports, images of the user stored as part of journal entries or separately, user date from other applications such as biometric data from smartwatches or other activity trackers, information on the last time the user took their medication and/or the user's prescribed medicament regimen, patient demographics or other info that can be used to uniquely identify the patient, proof of income and insurance coverage status.

In some embodiments a three tier consent system may be used. A first tier of consent may be used for a doctor. The first tier of consent may grant the highest level of access to sensitive user information. A second tier of consent may be used for a healthcare provider, such as a pharmacy or surgery. Some personal health information needs to be shared with the pharmacy or surgery, such as prescription information but not as much as with the doctor. A third tier of consent may be used for a pharmaceutical company who manufacture the patients medicament and/or a data provider, such as a company making an activity or health monitoring system. It may be necessary for the pharmaceutical company to gather basic identity information on the patients taking the medicament, but not to have access to any sensitive patient specific health data. Similarly, it may be advantageous for the user to be able to import activity or physiological data into the application from a third party source, which may require granting the third party source certain consents to interface with the application and to verify the identity of the user, but not to receive any other patient specific information from the application or related systems.

Each of the roles 206 may have an associated permission template specifying the type of data that the role can access by default. Having predefined roles and consent templates can greatly streamline and simplify the process of assigning appropriate consent levels to the various users of the system. In addition, a single user ID can be associated with multiple roles within the system. The templates may be defined using the roles described above. For example, the dashboard viewer role may have an associated dashboard viewer permission template which allows the users assigned to this role to see only anonymized analytic data which does not enable the patient to be uniquely identified. The pharmacist role may have an associated pharmacist permissions temple which allows users assigned to this role to see more sensitive patient information which allows the patient to be uniquely identified, as well as detailing their medical conditions, prescription and address. A physician template may have further permissions set by default, such as permission to view any summary reports generated via the journal. The physician template may not allow access to all information within the user journal, although the patient may choose to allow access to particular information.

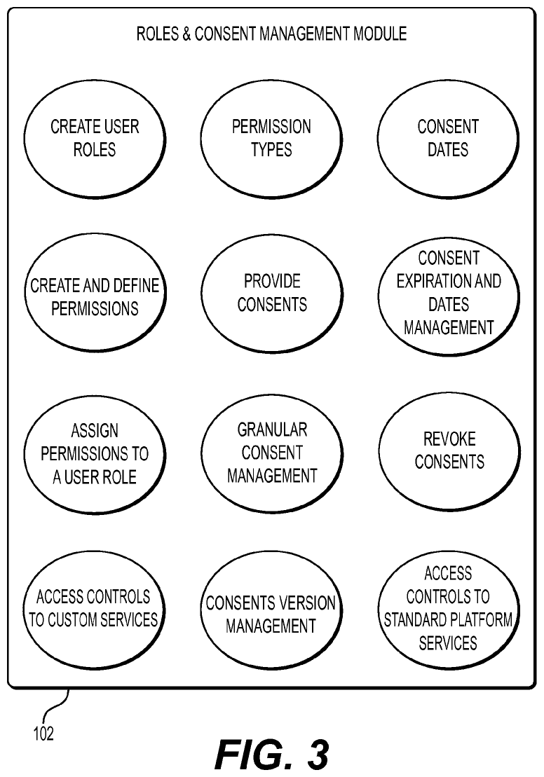

FIG. 3 is a diagram illustrating the functionality and stored information of the consent module 102. FIG. 3 shows twelve different aspects of consent management that the consent module 102 may store or be used to control. These are “Create user roles”, “Permission types”, “consent dates”, “Create and define permissions”, “Provide consents”, “Consent expiration and dates management”, “Assign permissions to a user role”, “Granular consent management”, “Revoke consents”, “Access controls to custom services”, “Consents version management” and “Access controls to standard platform services”. These categories are shown only as examples, the wording of which outlines the scope of the information being controlled via the consent module 102.

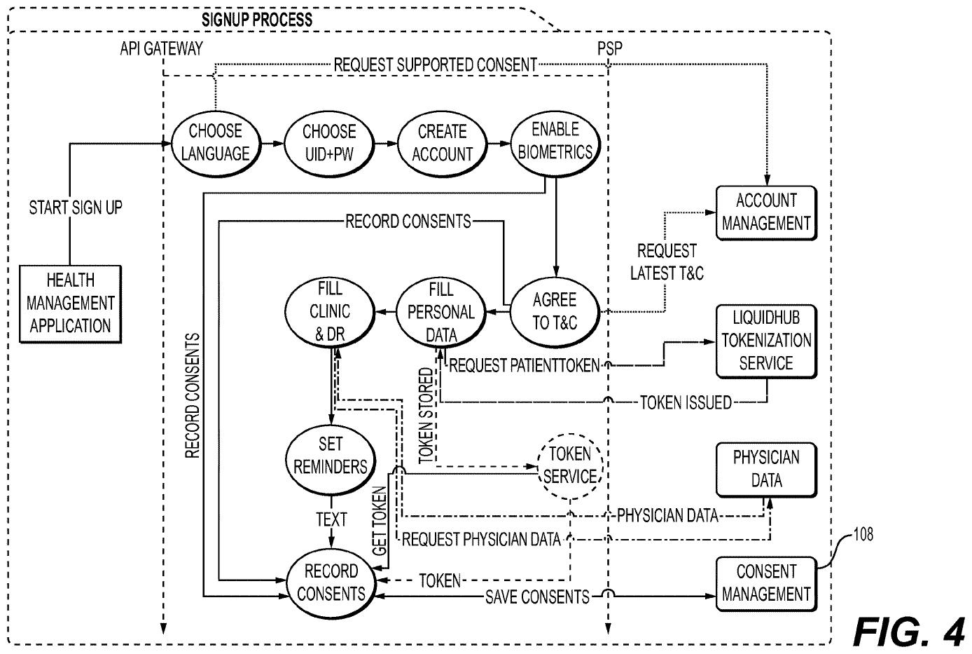

The consent module 102 and/or consent management service 108 may be queried and/or updated at various times during use of the application, as illustrated in the data flow diagrams of FIGS. 4 to 6.

FIG. 4 is a data flow diagram illustrating an application sign-up process. The patient opens the Health management application and follows a series of steps as indicated in the oval boxes to perform an initial set-up of the application. At several points during the set-up process the application may record the user's consents. Some of these may be internal consents relating to operation of the application, for example the application's permission to access the user's biometric information already on the user device 104 for unlocking the user device, or to access the user device camera, gallery and/or phone applications. At other points, the user is asked to agree to displayed terms and conditions. When the user enters personal information and/or health information as part of the sign-up process, they may then be asked for consent to store and share this information in accordance with a defined policy which is displayed to the user.

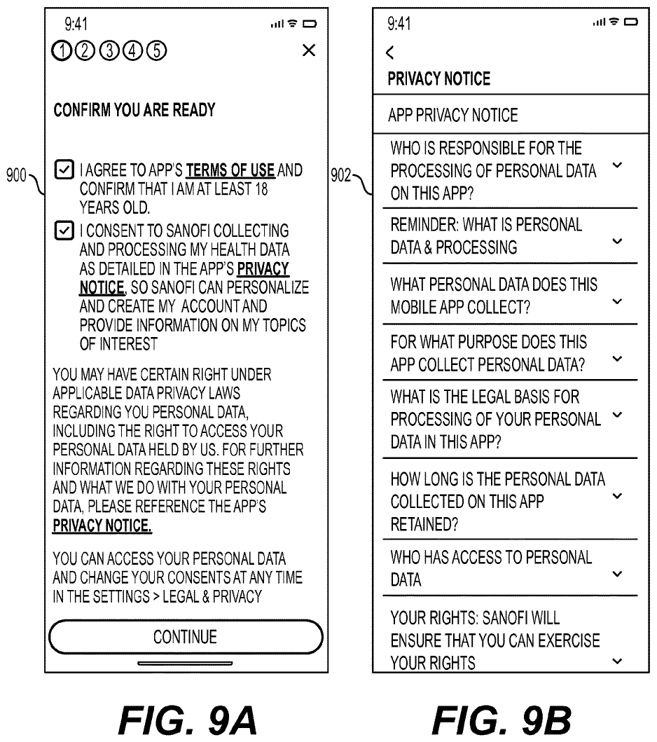

For example, a first consent screen 900 shown in FIG. 9a may be displayed during an account creation process and once the user has entered personal information and/or health information. The first consent screen 900 provides a link to a privacy notice and asks the user to give permission for their health data to be processed as detailed in this privacy notice. If the user selects the link to view the privacy notice, then the privacy notice screen 902 shown in FIG. 9b may be displayed by the application. The privacy notice screen 902 has a number of sections, which may be in the form of an FAQ. The user can select a section to expand it and view the information. Once the user's consents have been recorded, they are transmitted to the consent management service 108.

Several levels of user consent may be defined and may be asked for in relation to different actions the application may perform or different data the application may share with external systems. For example, a first level consent may be required to send the user marketing information, using the contact details they have provided during the sign-up process. A second level of consent may be required to allow the user to receive a call from a nurse as part of the onboarding process or a medicament administration training process. Neither the first or second levels of consent may require a signature of the user; the user may simply be presented with a consent statement and may tick a box or interact with a button to confirm their consent. A third level consent may be required to store the user's health related data on an external system and/or to share the user's health data with a pharmacy. The third level of consent may require the user's signature. A further level of consent may be required to combine the user's health information with the user's “consumer information”, such as the websites they visit and/or make purchases from. Combining this data may allow the application to provide more personalized content to the user.

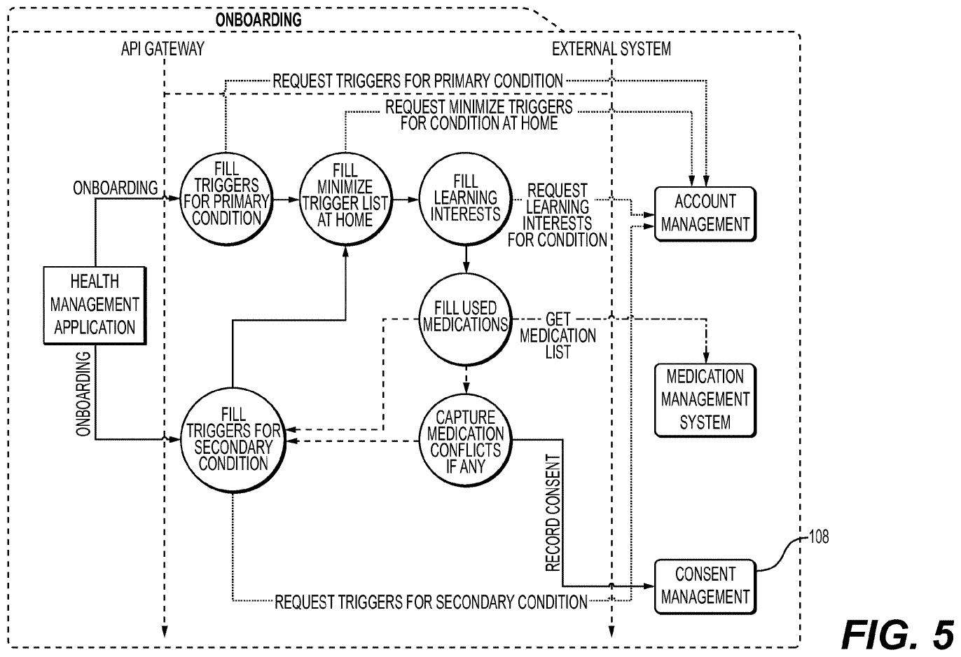

FIG. 5 is a data flow diagram illustrating an “onboarding” or application customization process performed by the user on the application. FIG. 5 illustrates the requests for user data input in the oval boxes in the center of the diagram and the movement of data (labelled arrows) between the application and external systems and services. An API gateway is programmed into the application for exchanging data with functional modules.

As part of the onboarding process, the user inputs information relating to trigger conditions for a primary condition, which may for example be Atopic Dermatitis. The user may then input what steps they currently take to minimize trigger conditions for the primary condition. The user may then indicate what topics they are interested in learning about in relation to their primary condition. The information entered by the user in each of these steps is communicated to an external account management service. The user may also input information on any other medications they are using and a check for conflicts may be performed.

At the end of the process, the application asks for the user's consent to store the information they have entered as part of the customization process. The user's consent information is communicated to the consent management service 108.

After collecting the information relating to the user's primary condition, the process may repeat in order to gather the same information in relation to the secondary condition, which may for example be Asthma. A separate consent request nay be presented in respect of the secondary condition, or the user's consent for all the information input may be requested at the end of the onboarding process.

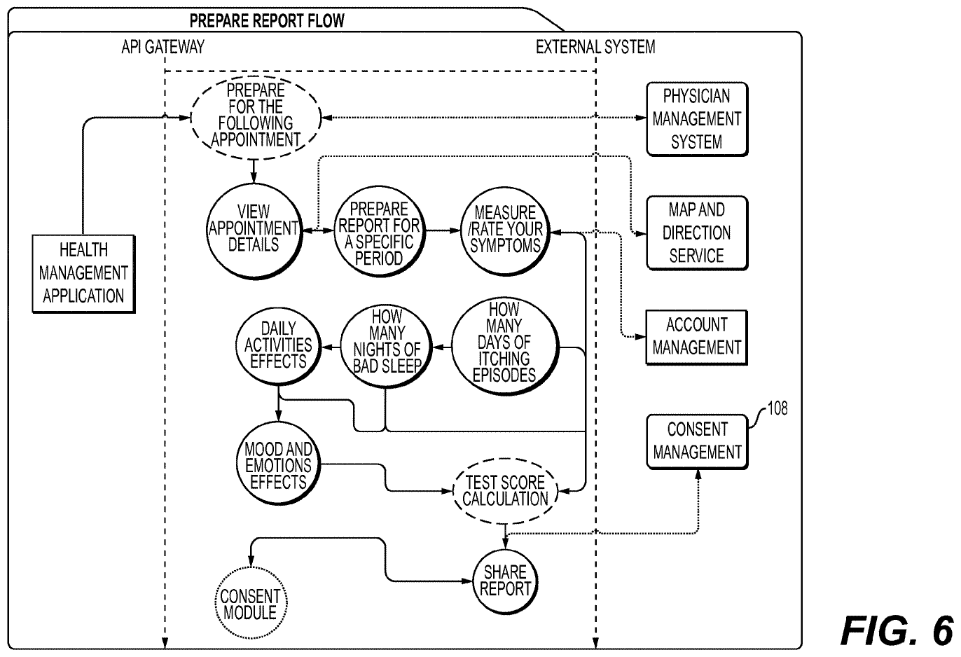

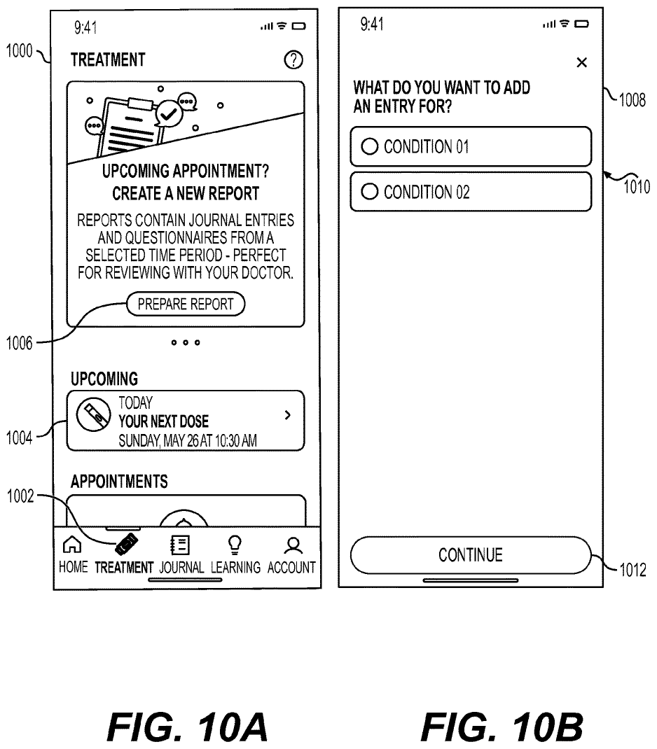

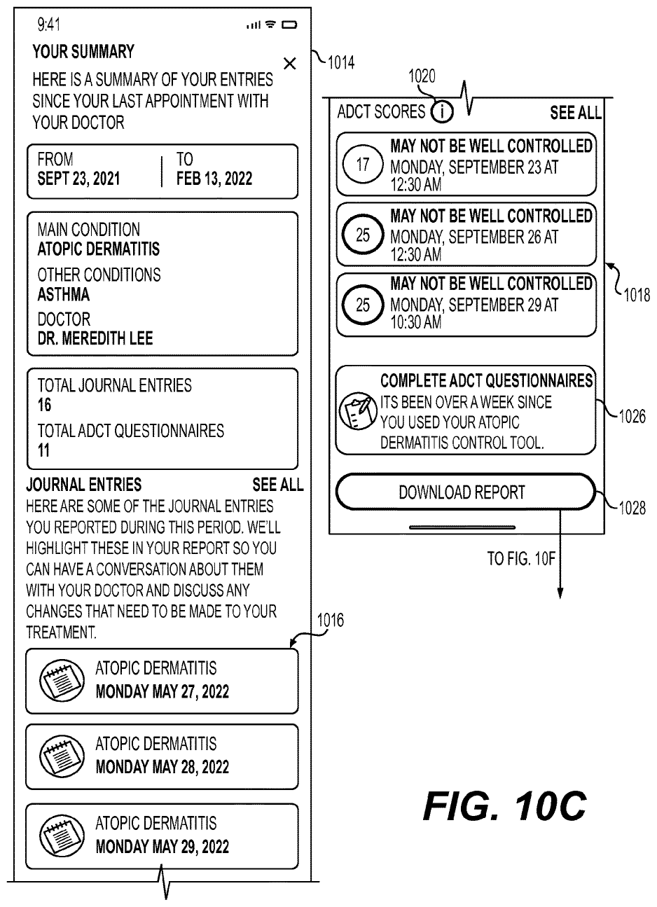

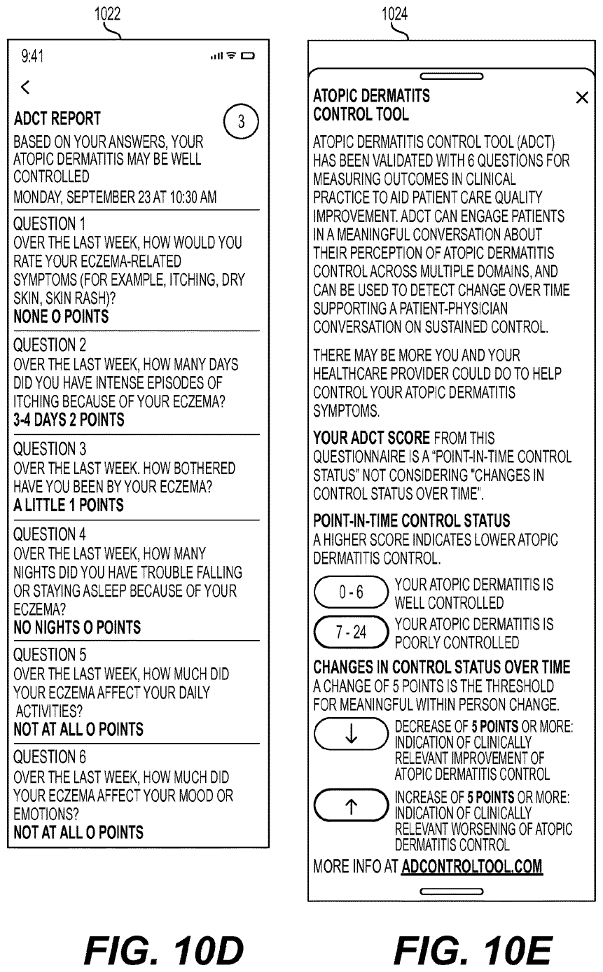

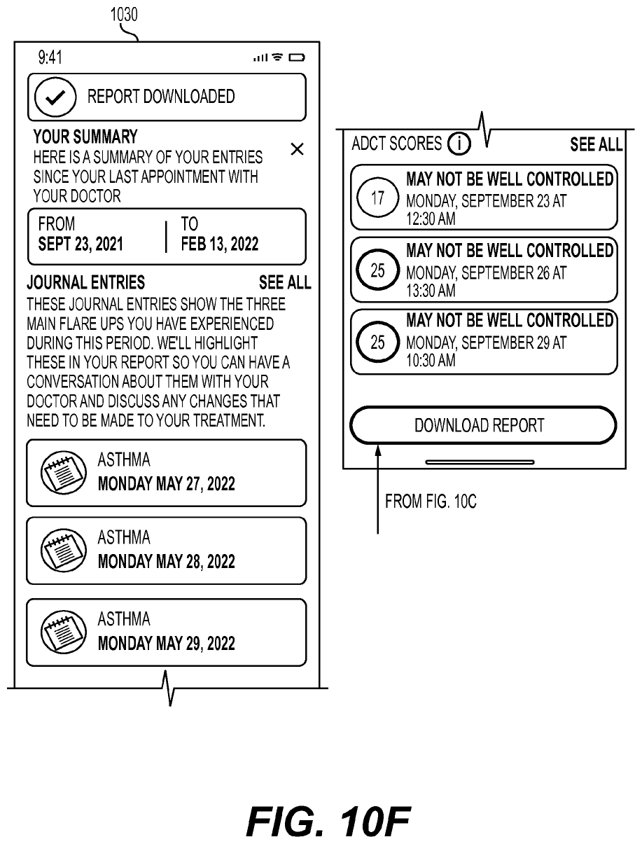

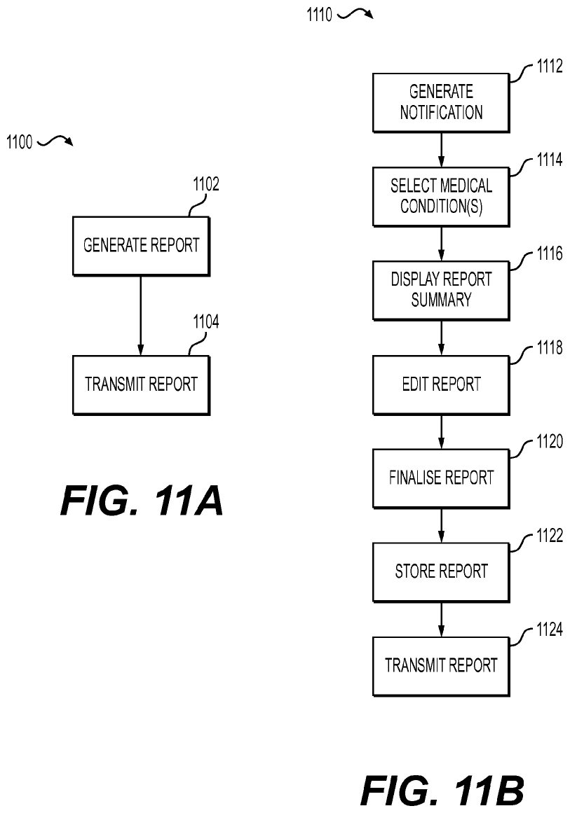

FIG. 6 is a data flow diagram illustrating steps involved in a report generation. Since this report contains user specific health information and is intended to be shared with the user's healthcare professional at least, some aspects of consent management are involved. The report generation involves the user inputting answers to various questions relating to their symptoms. This information results in the generation of a report which may also comprise a test score. The application may request that the user shares the generated report with their doctor or other healthcare professional, as described in greater detail below with reference to FIGS. 10a to 10f, 11a and 11b. If the user agrees to this request, the consent module 102 and/or consent management service 108 are queried to check that the indicated recipient has the appropriate permission to view the information.

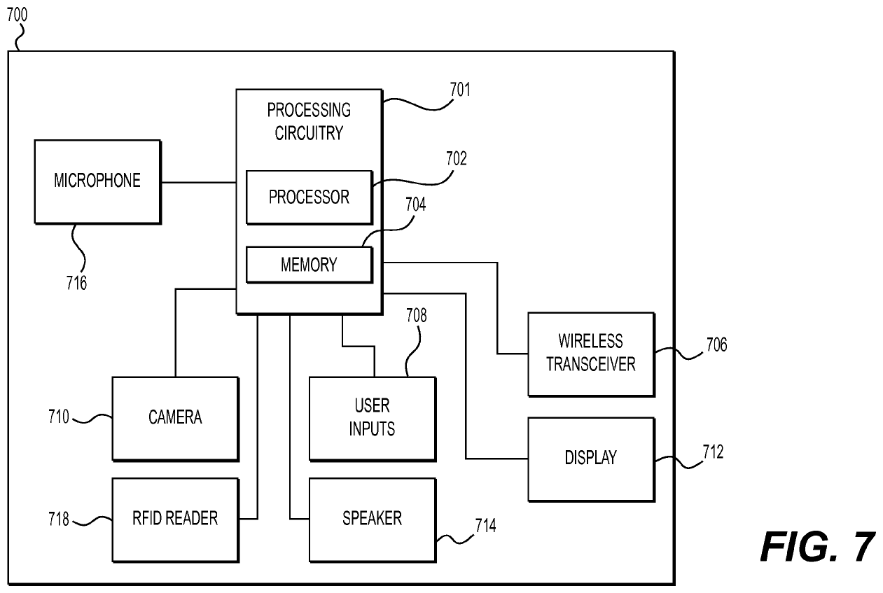

FIG. 7 illustrates schematically a user device 700 according to some embodiments. The user device 700 may be a mobile phone or tablet computer. The user device 700 is an example of a wireless communication device. The user device may alternatively be referred to as a communication device, computer, computing device or mobile device and is not necessarily associated with a single user. The user device 700 is configured to communicate wirelessly with a communications network using a wireless communication protocol, for example but not exclusively 3GPP LTE and/or New Radio (NR) or Wi-Fi (IEEE 802.11). The user device 700 may also be configured to communicate using Bluetooth, NFC, Zigbee, Ultra Wideband, IrDa or similar. The communications network may comprise one or more network nodes. The communications network may further be connected via a core network and/or an intermediate network to a host computer (not shown) which may be embodied in the hardware and/or software of a standalone server, a cloud-implemented server or a distributed server. The user device 700 and the host computer may be configured to communicate data.

The user device 700 may comprise hardware that includes processing circuitry 701. The processing circuitry 701 may comprise a processor 702 and a memory 704. The user device 700 may also comprise a wireless transceiver 706, user inputs 708, a display 712, a camera 710, a microphone 716, and RFID reader 718 and a speaker 714. The wireless transceiver 706 may be configured to set up and maintain a wireless connection to a network node. The wireless transceiver 706 may comprise one or more radio transmitters and one or more radio receivers. The display may be a touch sensitive display and may be based on capacitive or resistive sensing technology. The processing circuitry 701 may for instance include a microprocessor, a Digital Signal Processor (DSP), Application Specific Integrated Circuit (ASIC), Field Programmable Gate Array (FPGA) or the like. The processor 702 may be configured to read and/or write from the memory 704. The memory 704 may comprise a volatile and/or nonvolatile memory, for example a cache, RAM (Random Access Memory) and/or ROM (Read-Only Memory) etc.

The user device 700 may comprise software which is stored, for example, in memory 704. The software may be executable by the processing circuitry 701. The software may include an application. In some embodiments, the host computer may communicate with the application.

The application may request data from the host computer and/or provide user data to the host computer.

The processing circuitry 701 may be configured to perform or cause to be performed any of the methods described herein. In some embodiments, the software/program may include instructions that, when executed by the processing circuitry 701 cause the processing circuitry 401 to perform the methods described herein.

The memory 704 may comprise both a program memory storing program code (e.g. software or firmware) and main memory storing data. The processing circuitry 701 may be configured to execute the program code stored in the program memory and to read, write and delete data from the main memory. In some embodiments, the program code may be an application which can be downloaded and installed on the user device 700. The application may be a disease and treatment management and tracking tool for use by patients. The program memory may for instance be a Read-Only Memory (ROM), and the main memory may for instance be a Random Access Memory (RAM).

The user device 700 comprises one or more user inputs 708, for example a touchscreen, keypad or keyboard, accelerometer or gyroscope, mouse or microphone 716 for receiving voice commands. User device 700 may also comprise a camera 710 configured to capture images of a user and images of labels, codes and the like visible on the medicament administration devices, packaging or storage solutions. The user device 700 may be configured to scan medicament administration devices (such as an injection device or inhaler) using a scanning device. The scanning device may refer to either the camera 710 or RFID reader 418. The term “scanning” as used in relation to the user device 700 may refer to use of either of these components to read information provided externally or internally on medicament administration devices.

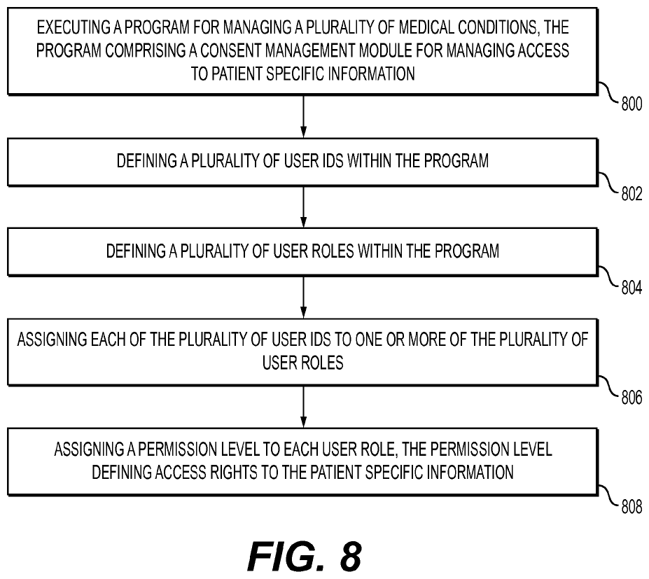

FIG. 8 shows a flow chart of an example method for defining and managing consent within the program for managing a plurality of medical conditions.

At operation 800, a program (also referred to as an application) for managing a plurality of medical conditions is executed on the computing apparatus/system. The program comprises a consent module for managing access to patient specific information. The patient specific information may be health related data, such as details of a user's prescription or reports and/or images of the user's disease progression. The user specific health information may comprise one more physiological measurements taken from the user. The physiological measurements may, for example, comprise one or more of: a heart rate; a blood pressure; a blood glucose level; a body temperature; a blood oxygen level or the like. The user specific health information may alternatively or additionally comprise one or more activity measurements, e.g. a step count, a sleep pattern, a distance travelled or the like. The user specific health information may alternatively or additionally comprise one or more dietary measurements, e.g. a calorie intake, fat intake and/or salt intake.

At operation 802, a plurality of user IDs are defined within the program. A patient ID may be defined during the sign-up process shown in FIG. 4. A user ID associated with the user's physician may also be defined during this set-up process.

At operation 804, a plurality of user roles are defined within the program. The user roles may be defined in the consent module 102 of the application. The user roles may for example include: patient, physician, guardian, pharmacist and dashboard viewer, among others.

At operation 806, each of the plurality of user IDs is assigned to one or more of the plurality of user roles. Some of the association may be made without the user's direct input. For example the user ID associated with the user/patient may automatically be associate with a predefined “User” role. In some other instances, the association may be done indirectly by, for example, asking the user to input details of their physician. This may create a user ID for the physician and automatically associate that user ID with a predefined “physician” role.

At operation 808, a permission level is assigned to each user role, the permission level defining access rights to the patient specific information. The user may be able to individually set the permission level in respect of each role defined in the system. In addition, the system may provide one or more consent templates linking one or more roles with predefined permission levels. For example, a consent template may be provided linking a default “physician” role to a permission level allowing the physician to access prescription details and reports generated by the application. The permission level may not allow access to some data, for example images stored within the application. A user may however manually change the permission level assigned to the “physician” role or create a new role and assign their physician's user ID to this new role.

In addition to the operations mentioned above, the application may also revoke a permission level assigned to a role. For example, the user may view the permission level assigned to each of the roles defined in the system and then revoke or downgrade the permission level. The user may also define a time limit related to an assigned permission level. For example, the user may define that a “physician” role has permission to access prescription details and reports generated by the application for a period of 6 months or 1 year. The application may automatically revoke the permission level assigned to the role when time limit is reached.

In addition, instead of assigning a permission level to a role, the user may use the application to assign a permission level to a particular user ID of the plurality of user IDs. For example instead of assigning the same permission level to all Healthcare professional via a “healthcare professional” role, the user may individually assign permission levels to their physician, pharmacist etc using the user ID for those entities. As before, the user may also define a time limit related to an assigned permission level for each user ID and the application may automatically revoke the permission level assigned to the user ID when time limit is reached.

The permission levels assigned by the user to roles or to particular user IDs may be checked when an external user request access to any patient specific information. An exemplary embodiment of how patient specific information is created and communicated to an external user, including checking of consent will now be described with reference to FIGS. 10a to 10f. FIGS. 10a to 10f show screenshots illustrating the application facilitating user preparation for an appointment with an HCP.

The application assists the user in creating a report and/or otherwise preparing for an upcoming appointment or call with a doctor, a nurse or other health care professional in which support on the management, treatment and/or progression of the user's medical condition or conditions is provided. Hereafter, the term health care professional is used to encompass all such professionals. The application prompts a user to prepare for an upcoming or requested appointment with an HCP. In particular, the application prompts a user to create a report and transmit that report to the HCP or an external device accessible by an HCP. The report may include patient information and/or information detailing the recorded treatment and/or disease progression recorded by the user.

In the first screenshot 1000 shown in FIG. 10a, a screen is displayed in which a treatment tab 1002 is selected. The application shows a number of user selectable tabs at the bottom of the screen, the tabs including at least two of Home screen, Treatment, Journal, Learning and Settings. The treatment screen has a notification area where silent notifications are shown. A first silent notification 1004 is shown reminding the user that their next dose is due today.

Alternatively the first silent notification may remind the user that they have an upcoming appointment with an HCP. Interacting with the first silent notification 1004 may open a notification center, where any further details relating to the notification can be seen as well as any other notifications which are awaiting the user's review. In the top portion of the home screen, a second silent notification or a graphic with a suggested action is displayed. The content of the suggested action changes depending on the proximity of a user's next scheduled dose, doctor's appointment, prescription renewal/delivery date, etc. In the example of the first screenshot 1000 of FIG. 10a, the suggested action is to prepare a report for an upcoming appointment. A user selectable option 1006 is provided prompting the user to prepare a report. When the user interacts with the user selectable option 1006, the second screenshot 1008 of FIG. 10b or the third screenshot 1014 of FIG. 10c is shown.

The second screenshot 1008 of FIG. 10b is shown if the application is assisting the user in the management and/or treatment of more than one disease. In other words, the second screenshot 1008 of FIG. 10b may be shown if the user is a comorbid user. The screen includes a plurality (two or more) of user selectable options 1010, each associated with a medical condition of the user. The user can elect to select one or more of these icons so that an entry for each of the selected medical conditions will be included in the report. When the user selects one or more of these icons, the user selectable option 1012 allowing the user to elect to continue may be activated or otherwise enabled to be selectable. The user may elect to continue with preparing the report, or to cancel the action. When the user interacts with the user selectable option 1012, the third screenshot 1014 of FIG. 10c is shown.

Alternatively, the third screenshot 1014 of FIG. 10c is shown when the application is assisting the user in the management and/or treatment of a single medical condition. In this example, when the user interacts with the user selectable icon 1006 shown in FIG. 10a, the second screenshot of FIG. 10b is omitted and the third screenshot 1014 of FIG. 10c is immediately shown.

In FIG. 10c, the third screenshot 1014 shows a report summary screen which displays a number of areas providing a variety of information including patient information and/or information detailing the recorded treatment and/or disease progression recorded by the user. For instance, the areas may include information detailing one or more of: the time period encompassed by the report, patient information, medical conditions, prescription information, and medical practice and/or doctor with which the user is registered. Other information may equally well be provided or omitted in various combinations. For instance, in the example shown in FIG. 10c, two medical conditions are shown, but the user may equally well have a single medical condition or more than two medical conditions. The report summary screen includes a Journal area where one or more user selectable icons 1016 representing journal entries are shown. Interacting with the user selectable icons 1016 may open the selected journal entry. The Journal area may also include a user selectable option (not shown) to complete a journal entry. The journal entry and the process of completing the journal entry will be discussed in more detail further below.

The report summary screen includes a Control Tool area where one or more user selectable icons 1018 representing a control tool questionnaire entry are shown. The control tool questionnaire provides a score associated with the control or progression of a user's medical condition(s). Interacting with the user selectable icons 1018 may open a report detailing the selected control tool questionnaire entry. An example report is shown in the fourth screenshot 1022 of FIG. 10d. The control tool area may also include a user selectable icon 1020 that provides information about the control tool questionnaire. The icon may be represented as an “i”, although other graphics may equally well be used. Interacting with the user selectable icon 1020 may open a screen including a control tool information area, such as the screen shown in the fifth screenshot 1024 of FIG. 10e. The control tool information area provides further information to explain the purpose and function of the questionnaire and score associated with the control tool. The screen may also display further links to provide the user with access to additional information regarding the tool. Referring back to FIG. 10c, the Control Tool area may also include a further user selectable option 1026 to complete a control tool questionnaire. In FIG. 10c, the report summary screen also includes a user selectable option 1028 to allow the user to elect to download the report. Interacting with the user selectable option 1028 provides confirmation from the user that the report is ready to be downloaded, and in response the application downloads the report. When the user interacts with the user selectable option 1028, the sixth screenshot 1030 of FIG. 10f may be shown. The sixth screenshot 1030 includes a graphic confirming that the report has been downloaded. The application records, saves or otherwise stores the report. The application may store the report in a memory of the user device. Alternatively, the application may be linked to the cloud, and the report may be stored remotely.

In response to the user electing to download the report, the application may automatically transmit the report. Before the report is transmitted, the permission level associated with the recipient is checked by the consent module 102 and/or consent management service 108, as shown in FIG. 6. If the recipient has the correct permission level to view the information contained within the report, then the report is automatically transmitted. If the recipient does not have the correct permission level, then the report is not transmitted. A warning may be displayed alerting the user to the lack of permission.

In another example, once the report has been downloaded, the application may only transmit the report in response to confirmation from the user electing to transmit the report to an HCP.

The application may display a notification prompting the user to elect to transmit (or not transmit) the report to an HCP. In response to a positive user interaction with the notification to elect to transmit the report, the application transmits the report to the HCP (and vice versa). The permission level of the HCP is still checked via the same process described above. The application may transmit the report to the HCP, either directly or via the cloud or other external device that can be accessed by the HCP. In response to a negative user interaction with the notification, the application does not transmit the report.

In some embodiments the report is transmitted to an external server or database. The HCP may then access the report from this external server or database. The permission level of the HCP may also be checked by the external server or database at the point at which they attempt to access the report.

Referring now to FIGS. 11a and 11b, a flow chart 1100 illustrating the appointment preparation process is shown in FIG. 11a, and a flow chart 1110 illustrating the appointment preparation process including some optional steps is shown in FIG. 11b.

In FIG. 11a, a report is generated at step 1102. The report is generated for an appointment with an HCP. The report may detail patient information and/or information detailing the recorded treatment and/or disease progression recorded by the user. For instance, the report may include one or more journal entries and/or control tool questionnaires.

The report may be generated in response to one or more different triggers. The report may be generated in response to a user input electing to generate the report. Alternatively and/or additionally, the report may be generated automatically in response to a scheduled appointment or a newly requested appointment with an HCP. For instance, the application may be arranged to monitor a current appointment schedule set within the application. From this, the application can determine when an appointment is scheduled. For instance, the application may determine that there is a scheduled appointment based on a calendar entry. The calendar entry may relate to one or more of a date, a day, or a time of an appointment. In some examples, the report may be generated in response to a scheduled reminder. The reminder can be associated with or independent from a scheduled appointment. The reminder may be set in advance by the user. In another example, the application may also determine a time remaining before an appointment. The time may be measured in minutes, hours or days. In response to determining that the time remaining before the appointment is less than a threshold time, the application generates the report. The threshold time may be measured in minutes, hours or days. The threshold time may be pre-set in the application or set by a user.

At operation 1104, the program transmits the report to an HCP. Once the report is generated, the report is transmitted to an HCP automatically or in response to an input from the user. The process of transmitting the report is substantially the same as that discussed above with respect to FIG. 10c, including the steps associated with checking the permission level of the HCP.

Referring now to FIG. 11b, at step 1112, a notification to prompt the user to prepare for an appointment with an HCP is generated. The appointment may be scheduled or newly requested by the HCP and/or user. In one example, the notification may be a push notification that is pushed to the lock screen or home screen of the user device. In another example, the notification may be a silent notification. The silent notification may be pushed into a screen silently and viewable only within that screen. The silent notification may be displayed on the screen as a reminder or suggestion to the user to prepare for an upcoming appointment with an HCP. For instance, the screen may be a home screen of the application. Alternatively or additionally, the screen may be a treatment screen 1002, as shown in the first screenshot 1000 of FIG. 10a.

The notification prompts the user to prepare for the appointment by preparing a report. The report may detail patient information and/or information detailing the recorded treatment and/or disease progression recorded by the user. The user can elect to prepare for the appointment with the HCP, by interacting with the notification. In response to interacting with the push notification, the application may open a new screen for preparing the report. For instance, the application may display the treatment screen shown in the first screenshot 1000 of FIG. 10a. The application can be arranged to display the notification in response to one or more different triggers. The triggers may be the same as those discussed above in respect of FIG. 11a, such that corresponding description shall be omitted.

The notification provided in step 1112 is optional and may be omitted. In some examples, the user may independently select to prepare for the appointment with the HCP by preparing a report without receiving a notification. For instance, the user may interact with the user selectable option 1006 shown in the first screenshot 1000 of FIG. 10a.

At step 1114, a user input to select one or more medical conditions to be included in the report is detected. For example, the user may select one or more of the user selectable options 1010, as shown in the second screenshot 1008 of FIG. 2b. This step is optional and may be omitted. For example, it may be omitted in the user has only one medical condition that is being treated.

At step 1116, a screen showing a summary of the report of the selected medical conditions is displayed. The report summary may correspond to the summary shown in the third screenshot 1014 of FIG. 10c. The report summary indicates the information to be included in the report. The report summary provides the user with the option to review and/or edit the information to be included in the report.

At step 1118, the user can select to edit the report. Step 1118 is optional and the user may elect not to edit the report. The user may edit the report by amending, adding or removing information. For instance, the user may add additional information by completing a journal entry and/or a control tool questionnaire for inclusion in the report. The user may complete a journal entry, for example, by selecting a user selectable option (not shown) from the screen in the third screenshot 1014 of FIG. 10c. The process of completing the journal entry may correspond to that described above.

Alternatively or additionally, the user may complete a control tool entry, for example, by selecting the user selectable option 1026 shown on the screen in third screenshot 1014 of FIG. 10c.

At step 1120, the report is completed or finalised. The report may be finalised, in response to detecting a user input to download the report. The user input may select the user selectable option 1028 shown on the screen in the third screenshot 1014 of FIG. 10c. The process of downloading the report is substantially the same as that described above with respect to FIG. 10c, such that corresponding description shall be omitted.

At step 1122, optionally, in response to detecting that the report is finalised, a record of the report is stored. The application may store the report in a memory of the user device.

Alternatively, the application may be linked to the cloud, and the report may be stored remotely. At step 1124, the application transmits the report to the HCP. The application may transmit the report automatically, for instance, in response to the user selection to the download the report. Alternatively, the program may transmit the report in response to a user input. The process of transmitting the report is substantially the same as that described above with respect to FIG. 10c, including the steps associated with checking the permission level of the HCP before access to the report is granted. The HCP may receive the report directly or may access the report via a remote database.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short-or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20 °C), or refrigerated temperatures (e.g., from about 4 °C to about 4 °C). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body. The drugs or medicaments contained in the drug delivery devices as described herein can be used for the treatment and/or prophylaxis of many different types of medical disorders.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen. Examples of antibodies are anti PCSK-9 mAb (e.g., Alirocumab), anti IL-6R mAb (e.g., Sarilumab), and anti IL-4R mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

1-20. (canceled)

21. A computer-implemented method comprising:
executing a program for managing a plurality of medical conditions, the program comprising a consent module for managing access to patient specific information;
defining a plurality of user identities (IDs) within the program;
defining a plurality of user roles within the program;
assigning each of the plurality of user IDs to one or more of the plurality of user roles;
assigning a permission level to each of the plurality of user roles, the permission level defining access rights to the patient specific information;
receiving a request to transmit the patient specific information to an external device, the request associated with a first user ID; and
in response to receiving the request:
determining the user role assigned to the first user ID; and
determining that the permission level assigned to the user role of the first user ID permits access rights to the requested patient specific information, and in response, transmitting the requested patient specific information to the external device.

22. The computer-implemented method of claim 21, wherein the method further comprises:
generating a notification prompting a user to prepare for an upcoming appointment; and
in response to receiving a user interaction with the notification, preparing a report comprising the patient specific information.

23. The computer-implemented method of claim 21, wherein the patient specific information is health related data.

24. The computer-implemented method of claim 21, further comprising revoking a permission level assigned to a first role of the plurality of user roles.

25. The computer-implemented method of claim 21, further comprising:
defining a time limit related to a permission level assigned to a first role of the plurality of user roles; and
revoking the permission level assigned to the first role of the plurality of user roles when time limit is reached.

26. The computer-implemented method of claim 21, further comprising assigning a permission level to a particular user ID of the plurality of user IDs.

27. The computer-implemented method of claim 26, further comprising:
defining a time limit related to assigning a permission level to the particular user ID, and
revoking the permission level assigned to the particular user ID when the time limit is reached.

28. The computer-implemented method of claim 21, further comprising providing a consent template linking a first role of the plurality of user roles with a predefined permission level.

29. The computer-implemented method of claim 21, wherein the plurality of user roles are selected from a list comprising at least two of patient, physician, guardian, pharmacist or dashboard viewer.

30. The computer-implemented method of claim 29, wherein a patient is deemed to be the sole owner of all patient specific information.

31. The computer-implemented method of claim 30, wherein the patient is the only user that can assign consent to other users and/or roles.

32. The computer-implemented method of claim 21, wherein the patient specific information is categorized into different sensitivity levels.

33. The computer-implemented method of claim 21, wherein the patient specific information comprises a plurality of resources and wherein the method further comprises assigning access rights to a subset of the plurality of resources and wherein assigning a permission level comprises granting access to the subset of the plurality of resources.

34. The computer-implemented method of claim 33, wherein a first resource of the plurality of resources comprises information identifying a user and a second resource of the plurality of resources comprises one or more journal entries.

35. The computer-implemented method of claim 33, wherein each resource comprises a plurality of information fields.

36. The computer-implemented method of claim 35, further comprising granting access to a subset of the plurality of information fields within a resource.

37. The computer-implemented method of claim 36, wherein the information fields comprise a username, a user address, a medicament and an injection device type.

38. One or more non-transitory computer-readable storage media storing instructions that, when executed by one or more computers, cause the one or more computers to:
execute a program for managing a plurality of medical conditions, the program comprising a consent management module for managing access to patient specific information;
define a plurality of user IDs within the program;
define a plurality of user roles within the program;
assign each of the plurality of user IDs to one or more of the plurality of user roles;
associate a permission level with each user role, the permission level defining access rights to the patient specific information;
receive a request to transmit the patient specific information to an external device, the request associated with a first user ID; and
in response to receiving the request:
determine the user role assigned to the first user ID; and
determine that the permission level assigned to the user role of the first user ID permits access rights to the requested patient specific information, and in response, transmitting the requested patient specific information to the external device.

39. The one or more non-transitory computer-readable storage media of claim 38, wherein the instructions further cause the one or more computers to:
generate a notification prompting a user to prepare for an upcoming appointment; and
in response to receiving a user interaction with the notification, prepare a report comprising the patient specific information.

40. A user device comprising one or more processors and one or more storage devices storing instructions that, when executed by the one or more processors, cause the one or more processors to:
execute a program for managing a plurality of medical conditions, the program comprising a consent management module for managing access to patient specific information;
define a plurality of user IDs within the program;
define a plurality of user roles within the program;
assign each of the plurality of user IDs to one or more of the plurality of user roles;
associate a permission level with each user role, the permission level defining access rights to the patient specific information;
receive a request to transmit the patient specific information to an external device, the request associated with a first user ID; and
in response to receiving the request:
determine the user role assigned to the first user ID; and
determine that the permission level assigned to the user role of the first user ID permits access rights to the requested patient specific information, and in response, transmit the requested patient specific information to the external device.
